(19) United States
(12) Patent Application Publication
(10) Pub. No.: US 2005/0222106 A1
(43) Pub. Date: Oct. 6, 2005

(54) Drospirenone-containing preparations for transdermal use

(76) Inventor: Stefan Bracht, Jena Cospeda (DE)

Correspondence Address: STRIKER, STRIKER & STENBY, 103 EAST NECK ROAD, HUNTINGTON, NY, 11743, US

(21) Appl. No.: 11/091,633

(22) Filed: Mar. 28, 2005

Related U.S. Application Data
(60) Provisional application No. 60/558,414, filed on Apr. 1, 2004.

(51) Int. Cl. A61K 31/57

(52) U.S. Cl. 514/177

(57) ABSTRACT

The pharmaceutical preparation for transdermal administration contains solvent ingredients, such as water and ethanol and/or propanol, and drospirenone. The drospirenone is contained in the preparation in an amount that is not above its saturation solubility in an initial state prior to application to skin. However after application to the skin the amount of drospirenone exceeds its saturation solubility due to escape or discharge of the solvent ingredients from the preparation. Preferably the saturation solubility is exceeded by at least a factor of five during application to the skin. The pharmaceutical preparation can also contain an estrogen, such as ethinyl estradiol. It can be in the form of a semi-solid or liquid preparation that is contained in a reservoir-type transdermal patch. A transdermal patch for contraception containing the pharmaceutical preparation including drospirenone and ethinyl estradiol is also disclosed.

### CROSS-REFERENCE

The present invention is also described in U.S. Provisional Patent Application 60/558,414, filed Apr. 1, 2004, which describes the same invention as described hereinbelow and provides basis for a claim of priority under 35 U.S.C. 119.

### BACKGROUND OF THE INVENTION

Steroid hormones are already currently in a large number of commercial products administered transdermally. This happens during hormone therapy in men and women and also for contraception in women.

In spite of the large number of transdermal medications there are only a small number of steroidal effective ingredients that are administered in this way. Some are from the estrogen group. For example, estradiol and ethinyl estradiol (e.g. ESTRADERM®, CLIMARA®, Fem7, Ortho-EVRA®, ESTRADOT®) are administered transdermally. The gestagens levonorgestrel, norethisterone, norethisterone acetate (e.g. Fem7 Combi, COMBIPATCH®) and norelgestromine (Ortho-EVRA®) are also administered transdermally. In andrology testosterone is available as a transdermal medication (e.g. ANDRODERM®, TESTODERM®, TESTOGEL®).

However the feasibility of transdermal application is not based on good skin transport for the steroid hormone, but only on its high effectiveness. Ethinyl estradiol and levonorgestrel are already effective at daily doses in a range of 20 to 50 μg per day. Norethisterone acetate belongs to the weaker effective ingredients with a typical transdermal daily dose of 125 to 250 μg per day required.

Among the gestagens used for therapeutic purposes dienogest and drospirenone belong to the group of less potent effective ingredients in regard to the required daily dosage, since they must be administered with a typical oral daily dose of 2 to 3 mg, in order to provide effective contraceptive action. In contrast to the so-called high potency gestagens, such as gestodene (see U.S. Pat. No. 5,788,984) or norelgestromine (Ortho-EVRA® Patch), which have effective daily dosages in the low two-digit microgram range, drospirenone and dienogest currently are considered to be unsuitable for transdermal application.

The single steroid hormone, which currently can be administered transdermally with a daily dosage in the milligram range, is testosterone. Generally the commercial patch products, TESTODERM® and ANRODERM®, are known for their small local compatibility or the little patient compliance because of the application site on the testicle sack (TESTODERM®) or the required patch size of 74 cm² (ANDRODERM®, two patches simultaneously applied). On the other hand, the gel preparation TESTOGEL® (outside of Germany it is called ANDROGEL®) is available, which similarly permits resorption of about 5 to 10 milligrams testosterone per day with a reduced local irritation potential and improved application comfort.

### SUMMARY OF THE INVENTION

It is an object of the present invention to provide preparations containing the less potent gestagen drospirenone, possibly in the form of a transdermal gel, in the required amount for transdermal administration, which can be transdermally administered.

According to the invention this object was attained with a pharmaceutical preparation for application to skin, which contains solvent ingredients and drospirenone in an amount such that the saturation solubility of the drospirenone is not exceeded in an initial state prior to application to the skin, but in other amounts such that the saturation solubility of the drospirenone is exceeded after application of the pharmaceutical preparation to the skin due to escape or discharge of the solvent ingredients from the preparation.

Surprisingly it was found that drospirenone permeated or penetrated mouse skin in vitro to approximately the same good extent as testosterone when it was applied in an aqueous ethanolic vehicle. From this result it can be concluded that drospirenone can be administered to humans with a dosage in the lower milligram range as with testosterone. This unexpected finding was probably due to the very high amount of super-saturation of the effective ingredient during drying of the vehicle after it was applied to skin. Of course the mechanism of the super-saturation is by evaporation, especially of the alcoholic component from the transdermal gel, lotion or spray, however preparations according to the invention have unexpectedly high super-saturation for drospirenone as proves suitable and practical.

### DETAILED DESCRIPTION OF THE INVENTION

In an especially preferred embodiment of the invention the transdermal preparation contains ethanol and water in addition to drospirenone. The effective ingredient drospirenone (DRSP) is present completely dissolved initially. An ethanol content of at least 60% (m/m), especially preferably even at least 65% (m/m) is provided in the gel in order to guarantee a suitable solubility of DRSP of preferably 0.2 to 2% (m/m), especially preferably of 0.5 to 1% (m/m).

Gel formers from the cellulose derivative group, especially hydroxymethyl cellulose, hydroxyethyl cellulose or hydroxypropyl cellulose, come into consideration besides the ionic gel formers based on polyacrylic acid (Carbomers or Carbopols, such as Carbopol 940, 941 or 980) because of the high content of alcohol. Permeation accelerators, lubricating agents and antioxidants come into consideration as additional additives.

In order to facilitate the super-saturation of the system according to the invention in drospirenone (DRSP), use of a group of low-molecular weight auxiliary substances is preferred. These auxiliary substances have a good solvating power for DRSP, and also either escape by high volatility or by an especially good skin permeability under application conditions, whereby the preparation becomes super-saturated in DRSP. Furthermore these auxiliary substances have a good toxicological compatibility during application on the skin. Preferred auxiliary substances are selected from the group consisting of ethanol, isopropanol (2-propanol), 1,2-propandiol (propylene glycol), dipropylene glycol, 1,3-butandiol, diethylene glycol monomethyl ether, diethylene glycol monoethyl ether, propylene carbonate and isopropylidene glycerol and monoterpene ingredients of etheric oils.

These solvating agents for DRSP escaping from the transderaml preparation by evaporation or transdermal absorption can if required be combined with one or more co-solvents, which are easily either evaporated or absorbed by the skin.

Reservoir-type transdermal systems are another preferred embodiment of the present invention. They have the same preferred and optional ingredients as described above for the transdermal gel preparations. The manufacture of the reservoir systems and their filling can take place according to the state of the art and is well known to those skilled in the art.

The super-saturation factors for semisolid gels, lotions, foams and sprays, which occur during application of the preparations, are determined according to the following procedures.

The determinations of the solubility of drospirenone in these preparations or in the residue produced by drying are performed on a semi-theoretical basis to simplify the experimentation and especially the analytical testing process. The semi-theoretical starting point is that only the liquid ingredients of the formulation are called upon for these considerations. The amount of gel-forming or film-forming or thickening polymers in the preparation is negligible in the case of gels, lotions and sprays. However these polymeric ingredients frequently influence analytical processing of these samples. A test solution comprising the liquid ingredients alone is called upon as a substitute for the above-described transdermal preparation within the scope of the present invention.

In specific the following mixture was tested as a substitute or representative of the formulation of the example 2 provided hereinbelow for the above-described purposes:

Test Example (Prior to Drying):

| | Parts (m/m) |
|---|---|
| Isopropyl myristate | 1.00 |
| Pure water | 23.55* |
| Ethanol 96% | 73.50 |

*sum of 20.00 g water plus 3.55 g of water that would be present from the 3.70 g of 0.1 N NaOH used in example 2. NaOH itself is not included, since it is significant only when the polymer ingredients are present and it would provide a strongly alkaline reacting medium.

The determination of the solubility of drospirenone in the test solution prior to drying is performed as follows:

About 10 g test solution, exactly weight out, is mixed with drospirenone with stirring by a magnetic stirring device, until a clearly visible precipitate or sediment occurs. This solution is stirred for at least 24 hours further. If the precipitate is not visible after the 24 hours stirring, more drospirenone is added and the process is repeated. After finishing the stirring process about 1.0 ml test solution is removed and transferred into a 1.5 ml auto-sampler container with a screwed on cover. The sample is subsequently centrifuged for 10 minutes at at least 3000 G in this container, in order to precipitate any remaining undissolved drospirenone in the sample. The concentration of drospirenone is then measured with a suitable HPLC method in an aliquot of the obtained supernatant liquid. The measured value of the saturation solubility of the drospirenone in the test mixture obtained prior to drying in this way is expressed as DRSP_{S1} in mg/ml.

For the preparations according to the invention the conditional DRSPᵢₙᵢₜ is less than DRSP_{S1}, wherein DRSPᵢₙᵢₜ is the initial concentration of the drospirenone in the preparation prior to application and drying.

The determination of the solubility of drospirenone in the liquid test mixture after drying occurs as follows:

The test solution is spread in a layer with a thickness of about 50 μm on a flat inert carrier or support and is allowed to dry at about 32° C. (skin surface temperature) under ambient conditions for a definite time. The time interval for drying depends on the application duration. For a one-time one-day application it corresponds to 24 hours. It can also be a shorter time, when the degree of drying should be determined for a shorter time than the application time. The layer thickness of 50 μm is defined as the standard layer thickness for transdermal application of gels, lotions or sprays for the current applications of this invention. In the case of commercial testogels e.g. 5 ml of the gel is applied to the upper arm. A theoretical application area of about 90 cm² (given a gel density of about 0.9 g/cm³) results with the defined standard layer thickness of 50 μm.

In the case of the above-described test example about 4.36 g of test solution is exactly weighed out (weight=G_{P1}) and spread out in a tared Petri dish with a 10 cm diameter (corresponds to 78.53 cm²) so that a layer thickness of about 50 μm results. This starting material is allowed to dry at about 32° C. for a time interval of 24 hours on a hot plate (corresponds to a one time daily application). The amount of test solution and diameter of the dish is exemplary and non-limiting. The amount can be varied within the above-described description of the invention. The test mixture is not mechanically moved or mixed during testing but may rest quietly. There is no special air circulation over the test sample. At the test end the weight of the dried residue from the test solution is determined (weight=G_{P2}). From the obtained values the drying factor F_{E} for the test solution is calculated according to equation (1):
F_{E}=G_{P1}/G_{P2} (1).

About 250.0 g of test solution (exactly weighed out—weight G_{R1}) with the composition prior to drying are present in a tared 500 ml wide-mouth flask. Subsequently this starting material is evaporated in a rotary evaporator at about 32° C. under low pressure, until the remaining quantity of the preparation is in the same weight ratio relationship to the initial amount, as was found for the drying in the Petri dish over the time interval, until the amount remaining in the flask (weight=G_{R2}) fit the following equation (2):
G_{R2}=G_{R1}/F_{E} (2).

The increased starting sample of the test solution in the rotary evaporator is used to obtain a sufficiently large residual amount for the subsequent determination of the solubility of drospirenone. It is again processed at about 32° C. in order to reproduce the evaporation conditions on the skin qualitatively with as little adulteration as possible, but quantitatively more rapidly.

1.0 g is taken from the finally obtained residual amount in the flask and transferred into a 1.5 ml auto-sampler container with a screw cover. 100 mg drospirenone are added to this residual amount. Subsequently this starting material is treated with ultrasonic waves for an hour. After that the sample remain under ambient conditions for 24 hours. The sample is subsequently centrifuged for 10 minutes at at least 3000 G, in order to precipitate undissolved drospirenone to the bottom of the sample. The concentration of drospirenone in an aliquot of the supernatant is then determined with a suitale HPLC method. The obtained value corresponds to the saturation solubility of drospirenone in the dried test solution, DRSP_{S2} in mg/ml. In the case that less than 1 gram can be taken from the residual amount in the flask, typically with F_{E}>200, the starting material in the rotary evaporator can be correspondingly increased or the subsequent determination is reduced, for example to 50 mg drospirenone in 0.5 g dried test solution.

The super-saturation factor F_{SS} relates to drospirenone in the test solution is calculated according to equation (3) below during the drying:
F_{SS}=DRSPᵢₙᵢₜ[mg/ml]×F_{E}/DRSP_{S2}[mg/ml] (3),
wherein the initial concentration of drospirenone in the preparation is given as DRSPᵢₙᵢₜ [mg/ml].

Reference is made to the super-saturation factor F_{SS} in the claims of this patent application. A value for F_{SS} of 5 means, for example, that the saturation solubility of drospirenone during usage of the preparation is exceeded by about a factor of five.

In reservoir-type transdermal patches the solubility of drospirenone in the formulation of the effective ingredient reservoir is considered. A semi-theoretical starting point is again used. A test solution consisting of only the liquid ingredients, which represents the actual semisolid reservoir preparation, is tested. The saturation solubility in the reservoir preparation is determined on this basis analogous to the determination of DRSP_{S1} in mg/ml in gels, lotions and sprays (s.o.).

The drying factor F_{E} is a step here in the determination of the concentration factor F_{C}, since the reservoir dries by transdermal permeation of liquid ingredients. The effective surface of the reservoir system is covered with this foil and the reservoir system is weighed in this arrangement (G_{RS1}).

After that the reservoir system is exposed to pure water as acceptor liquid for a predetermined time interval at a temperature of 32° C. The predetermined time interval amounts to 24 hours for a system for a single daily application. After that the system is weighed (G_{RS2}). The drying factor FE in the reservoir preparation determined from the measured weight loss is given by equation (4):
F_{E}=G_{RF}/{G_{RF}−(G_{RS1}−G_{RS2})} (4),
wherein G_{RF} is the weight in the effective ingredient containing reservoir formulation contained prior to the test in the reservoir system.

The determination of the saturation solubility of drospirenone in the concentrated reservoir formulation (DRSP_{S2}) and the super saturation factor F_{SS} are performed analogously to the embodiments in the form of gels, lotions and sprays with equation 1, but wherein F_{C} replaces F_{E}.

### BRIEF DESCRIPTION OF THE DRAWING

The objects, features and advantages of the invention will now be illustrated in more detail with the aid of the following description of the examples of the preparation according to the invention, with reference to the accompanying figures in which:

FIG. 1 is a graphical illustration showing the dependence of average permeation values of testosterone and drospirenone as a function of time after application of the transdermal preparation of example 1 according to the invention to bare mouse skin; and

FIG. 2 is a graphical illustration showing the dependence of average permeation values of testosterone and drospirenone as a function of time after application of the transdermal preparation of example 1 according to the invention to human skin.

The following examples serve to further illustrate the claimed invention, but their details should not be considered as limiting the appended claims.

### EXAMPLES

### Example 1

### Hydro-ethanolic Gel

| | Parts (m/m) |
|---|---|
| Drospirenone | 1.0 |
| TESTOGEL ® | 99.0 |

The drospirenone is mixed directly with commercially obtained TESTOGEL® and completely dissolved in it.

The TESTOGEL® corresponds in its composition to ANDROGEL®. These compositions, which were given in Table 5 of U.S. Pat. No. 6,503,894, are described as follows:

TABLE 5
Composition of AndroGel.RTM.

| | Amount (w/w) | |
|---|---|---|
| | PER 100 g of | |
| SUBSTANCE | GEL | |
| Testosterone | 1.0 | g |
| Carbopol 980 | 0.90 | g |
| Isopropyl myristate | 0.50 | g |
| 0.1 N NaOH | 4.72 | g |
| Ethanol (95% w/w) | 72.5 | g* |
| Purified water (qsf) | to 100 | g |

*corresponds to 67 g ethanol

### Example 2

### Hydro-ethanolic Gel on Polyacrylate Basis

| | Parts (m/m) | |
|---|---|---|
| Drospiranone | 1.0 | |
| Ethinyl estradiol | 0.1 | |
| Isopropyl myristate | 1.0 | |
| Carbopol 940 | 0.7 | |
| 0.1 N NaOH | 3.7 | |
| Purified water | 20 | |
| Ethanol 96% | to 100 | g |

The preparation is made in a manner that is known to those skilled in the art by dissolving all ingredients and subsequent neutralization of the gel former with dilute sodium hydroxide, so that the gel formation sets in. Isopropyl myristate serves as a lubricating agent for the skin. The super-saturation in drospirenone occurs during application by evaporation of ethanol.

### Example 3

### Hydro-ethanolic Gel on Cellulose Basis with Crystallization Inhibiting Additive

| | Parts (m/m) | |
|---|---|---|
| Drospiranone | 0.5 | |
| Ethyl oleate | 2.0 | |
| Collidone 12 PF | 0.5 | |
| Hydroxyethyl cellulose | 1.0 | |
| Purified water | 30.0 | |
| Ethanol 96% | to 100 | g |

The preparation is made in a manner that is known to those skilled in the art by adding the gel former methyl cellulose to alcohol and water and subsequently adding the auxiliary and effective ingredient. Ethyl oleate acts as a permeation accelerator. The super-saturation in drospirenone occurs during application by evaporation of ethanol.

### Example 4

### Transdermal Patch of the Type Having a Reservoir System

| | | |
|---|---|---|
| Dropirenone | 5.0 | mg* |
| Ethanol 96% | etwa 196 | μl |
| ANDRODERM ® 2.5 mg patch | 1 | Piece |

The protective foil is removed from the above-described exactly weighed out ANDRODERM® patch. The contents of the now open membrane evaporate or escape under room conditions until about 180 mg are evaporated. This pre-treatment is required in order to provide sufficient room in the reservoir for the above-described added DRSP solution.

100 mg of drospirenone are dissolved in 4 ml of ethanol 96% under action of ultrasound. About 500 μl of this solution are drawn into a 1 ml one-time use or disposable syringe with a connected steel hollow needle (0.90×40 mm). The syringe is inserted into the reservoir and about 200 μl of this solution are injected with it. This is possible by cutting or punching in an additional air hole at the point, at which an air bubble is found in the semisolid reservoir. After that both air holes are sealed with a piece of teas-film. The reservoir contents are mixed by careful kneading with the fingers for a few minutes. Until the experimental use the system is equilibrated at least 24 hours under room conditions.

The super-saturation of drospirenone occurs during application of the system substantially by the transdermal resorption of ethanol, which occurs substantially faster than that of drospirenone.

### Example 5

### Transdermal Spray

| | Parts (m/m) |
|---|---|
| Drospiranone | 0.5 |
| Ethinyl estradiol | 0.1 |
| Oleyl alcohol | 1.0 |
| 1,2-Propandiol | 0.5 |
| Ethanol 96% | 60.0 |
| Dimethyl ether | To 100.0 |

The supersaturation in drospirenone occurs during application substantially by evaporation of ethanol. The dimethyl ether acts as propellant for the spray.

### Example 6

### Permeation of Mouse Skin by DRSP from the Preparation of Example 1

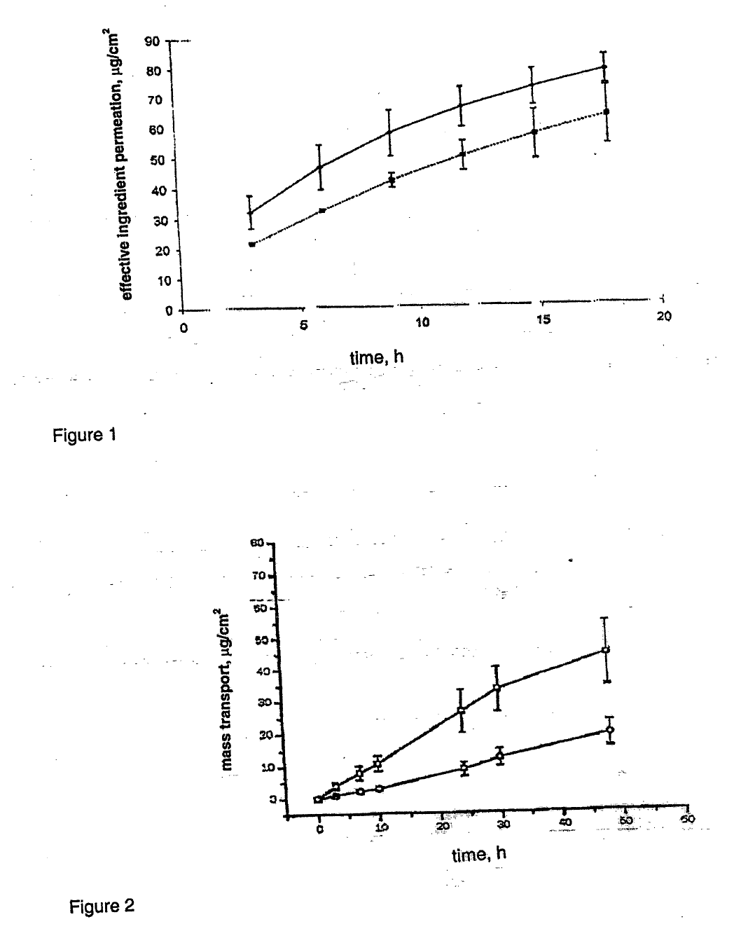

200 mg of the preparation made according to Example 1 were applied to bare mouse skin, which were fixed in modified Franz permeation cells as a permeation membrane. The results for the average permeation values for testosterone and drospirenone in μg/cm² versus time after application are shown in FIG. 1. The average values are the average of seven experimental runs. The mouse skin used was hairless mouse skin, HsdCpb NMRI-nu/nu, Harlan Bioservice, Walsrode.

Acceptable Medium for the Franz Cells:

| | | |
|---|---|---|
| Potassium chloride | 0.6 | g |
| Potassium hydrogen phosphate | 0.09 | g |
| Sodium chloride | 10.905 | g |
| Sodium hydrogen phosphate dehydrate | 0.09 | g |
| HEPES | 8.94 | g |
| Gentamicine sulfate | 0.075 | g |
| γ-cyclodextrin | 7.5 | g |
| Aqua purificata | to 1500 | g |

At 3, 6, 9, 12, 15 and 18 hours samples of 100 μl were removed by an automatic sampling system from the acceptor medium of the Franz cells and injected into an HPLC automatic analyzer.

HPLC Method:

| | |
|---|---|
| Column: | LiChrosphere 100 RP 18, 5 μm, |
| | 125 × 3 mm |
| Mobile Phase: | Acetonitrile/H₂O (40/60) |
| Flow Rate: | 1 ml/min |
| Temperature: | 40° C. |
| Injected volume: | 100 μl |
| Detection wavelength (UV): | 244 nm (testosterone), 261 nm (DRSP) |

The filled-in circles indicate the measured permeation values for testosterone in FIG. 1. The filled-in squares indicate the permeation values for drospirenone. The vertical lines on the circles and squares are error bars indicating the experimental error. Thus FIG. 1 clearly shows that the permeation values for testosterone are larger than those for drospirenone.

### Example 7

### Permeation of Human by DRSP from the Preparation of Example 1

The preparation from example 1 was tested on human skin (female belly skin from cosmetic reduction, dermatomized at a layer thickness of about 450 μm).

The skin permeation values or mass transport amount for drospirenone and testosterone as a function of time after application of the preparation to the skin are shown in FIG. 2. The open squares indicate the measured permeation values for testosterone in FIG. 1. The open circles indicate the permeation values for drospirenone. The vertical lines on the circles and squares are error bars indicating the experimental error.

This permeation data for human skin is also tabulated in the following table I and II.

TABLE I
PERMEATION OF TESTOSTERONE FROM THE PREPARATION
OF EXAMPLE 1 THROUGH HUMAN SKIN

| | Mean cumulative | | |
|---|---|---|---|
| Time, h | Transport, μg/cm² | Standard Deviation, SD | RSD, %* |
| 0 | 0.00 | 0.00 | 0 |
| 3 | 3.62 | 1.24 | 34 |
| 7 | 7.72 | 2.21 | 29 |
| 10 | 10.70 | 2.31 | 22 |
| 24 | 26.57 | 6.72 | 25 |
| 30 | 33.31 | 7.06 | 21 |
| 48 | 44.35 | 10.03 | 23 |

*RSD, % = (SD/mean) × 100.0

TABLE II
PERMEATION OF DROSPIRENONE FROM THE PREPARATION
OF EXAMPLE 1 THROUGH HUMAN SKIN

| | Mean cumulative | | |
|---|---|---|---|
| Time, h | Transport, μg/cm² | Standard Deviation, SD | RSD, %* |
| 0 | 0.00 | 0.00 | 0 |
| 3 | 0.87 | 0.34 | 39 |
| 7 | 2.04 | 0.68 | 33 |
| 10 | 2.86 | 0.75 | 26 |
| 24 | 8.67 | 2.19 | 25 |
| 30 | 12.13 | 2.64 | 22 |
| 48 | 19.23 | 4.03 | 21 |

*RSD, % = (SD/mean) × 100.0

### CONCLUSION

The foregoing permeation data shows that the permeation values for drospirenone (DRSP) are still about 40% of those of testosterone at the given times after application of the preparation to the skin. Since TESTOGEL® permits administration of 5 to 10 mg per day, then about 3 to 5 mg/d of drospirenone can be administered in this form by a transdermal method or patch. Thus the transdermal administration of an effective amount of DRSP for contraceptive purposes is possible.

While the invention has been illustrated and described as embodied in drospirenone-containing preparations for transdermal use, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A pharmaceutical preparation for application to skin, said pharmaceutical preparation containing solvent ingredients and drospirenone, said drospirenone being present in the preparation in an amount such that a saturation solubility of the drospirenone is not exceed in the preparation in an initial state of the preparation prior to application of the preparation to the skin, but wherein said drospirenone is present in the preparation in other amounts such that the saturation solubility of the drospirenone is exceeded after application of the preparation to the skin because of escape or discharge of the solvent ingredients from the preparation.

2. The pharmaceutical preparation as defined in claim 1, wherein the saturation solubility of the drospirenone is exceeded by a factor of at least five during application to the skin.

3. The pharmaceutical preparation as defined in claim 2, wherein the saturation solubility of the drospirenone is exceed by a factor of at least 2 after one hour after application to the skin.

4. The pharmaceutical preparation as defined in claim 1, wherein the saturation solubility of the drospirenone is exceeded by a factor of at least 10 during application to the skin.

5. The pharmaceutical preparation as defined in claim 4, wherein the saturation solubility of the drospirenone is exceed by a factor of at least 5 after one hour after application to the skin.

6. The pharmaceutical preparation as defined in claim 1, wherein the solvent ingredients comprise ethanol and/or isopropanol.

7. The pharmaceutical preparation as defined in claim 1, further comprising an inhibiting additive for preventing crystallization of the drospirenone.

8. The pharmaceutical preparation as defined in claim 7, wherein the inhibiting additive comprises soluble polyvinyl pyrrolidone.

9. The pharmaceutical preparation as defined in claim 1, further comprising at least one permeation accelerant.

10. The pharmaceutical preparation as defined in claim 1, in the form of a semi-solid composition and said semi-solid composition consists of an alcohol-containing gel.

11. The pharmaceutical preparation as defined in claim 1, in the form of a liquid alcohol-containing composition and wherein said liquid alcohol-containing composition consists of a lotion, foam or a spray.

12. The pharmaceutical preparation as defined in claim 1, in the form of a liquid or semi-solid alcohol-containing composition, which is part of a reservoir-type transdermal patch.

13. The pharmaceutical preparation as defined in claim 12, wherein the saturation solubility of the drospirenone is exceeded by a factor of at least 1.5 during application to the skin.

14. The pharmaceutical preparation as defined in claim 12, wherein the saturation solubility of the drospirenone is exceeded by a factor of at least 2 during application to the skin.

15. The pharmaceutical preparation as defined in claim 12, wherein said liquid or semi-solid alcohol-containing composition has an alcohol content of at least 60% m/m.

16. The pharmaceutical preparation as defined in claim 12, wherein said liquid or semi-solid alcohol-containing composition has an alcohol content of at least 65% m/m.

17. The pharmaceutical preparation as defined in claim 1, further comprising an estrogen compound.

18. The pharmaceutical preparation as defined in claim 17, wherein said estrogen compound is present in an amount such that a saturation solubility of the estrogen compound is not exceeded in the preparation in the initial state prior to the application to the skin, but in an amount such that the saturation solubility of the estrogen compound is exceeded after application of the preparation to the skin due to escape or discharge of the solvent ingredients from the preparation.

19. The pharmaceutical preparation as defined in claim 18, wherein said estrogen compound is ethinyl estradiol.

20. A reservoir-type transdermal patch for contraception, said reservoir-type transdermal patch comprising a pharmaceutical preparation for transdermal administration, wherein said pharmaceutical preparation contains solvent ingredients and drospirenone, and said drospirenone is present in the preparation in an amount such that a saturation solubility of the drospirenone is not exceed in the preparation in an initial state of the preparation prior to application to the skin, but wherein said drospirenone is present in the preparation in other amounts such that the saturation solubility of the drospirenone is exceeded after application to the skin because of escape or discharge of the solvent ingredients from the preparation.

21. The reservoir-type transdermal patch as defined in claim 20, wherein the saturation solubility of the drospirenone is exceeded by a factor of at least 1.5 during application to the skin.

22. The reservoir-type transdermal patch as defined in claim 20, wherein the solvent ingredients comprise ethanol and/or isopropanol.

23. The reservoir-type transdermal patch as defined in claim 20, wherein the pharmaceutical composition includes an estrogen compound and wherein said estrogen compound is present in an amount such that a saturation solubility of the estrogen compound is not exceeded in the preparation in the initial state prior to the application to the skin, but in an amount such that the saturation solubility of the estrogen compound is exceeded after application to the skin due to escape or discharge of the solvent ingredients from the preparation.

24. The reservoir-type transdermal patch as defined in claim 20, wherein the estrogen compound is ethinyl estradiol.
